(19) United States
(12) Patent Application Publication
(10) Pub. No.: US 2012/0107879 A1
(43) Pub. Date: May 3, 2012

(54) METHODS FOR PRODUCTION AND PURIFICATION OF NUCLEIC ACID MOLECULES

(75) Inventors: Christian E. GRUBER, Frederick, MD (US); Joel A. Jessee, Mount Airy, MD (US)

(73) Assignee: Life Technologies Corporation, Carlsbad, CA (US)

(21) Appl. No.: 13/220,433

(22) Filed: Aug. 29, 2011

Related U.S. Application Data
(63) Continuation of application No. 12/491,214, filed on Jun. 24, 2009, now abandoned.
Application No. 12/491,214 is a continuation of application No. 10/816,886, filed on Apr. 5, 2004, now abandoned.
Application No. 10/816,886 is a continuation of application No. 09/076,115, filed on May 12, 1998, now abandoned.
(60) Provisional application No. 60/046,219, filed on May 12, 1997.

(51) Int. Cl. C12P 19/34; C09K 3/00; C07H 21/04; C07H 21/00; C07H 21/02

(52) U.S. Cl. 435/91.3; 435/91.5; 435/91.51; 536/24.33; 252/183.11

(57) ABSTRACT

The present invention is directed to methods for the production and isolation of nucleic acid molecules. In particular, the invention concerns isolation of mRNA molecules and the production and isolation of nucleic acid molecules (e.g., cDNA molecules or libraries), which may be single- or double-stranded. Additionally, the invention concerns selection and isolation of particular nucleic acid molecules of interest from a sample which may contain a population of molecules. Specifically, the invention concerns affinity-labeled primer-adapter molecules which allow improved isolation and production of such nucleic acid molecules, increasing both product recovery and speed of isolation.

### CROSS REFERENCE TO RELATED APPLICATIONS

This application claims the benefit of U.S. Provisional Application No. 60/046,219, filed May 12, 1997, the disclosure of which is incorporated herein by reference in its entirety.

### FIELD OF THE INVENTION

The present invention is in the fields of molecular and cellular biology. The invention is particularly directed to methods useful for the production and isolation of nucleic acid molecules. In particular, the invention concerns isolation of mRNA molecules and the production and isolation of cDNA libraries (single- and double-stranded). Additionally, the invention concerns selection and isolation of particular nucleic acid molecules of interest from a sample which may contain a population of molecules. Specifically, the invention concerns the use of affinity-labeled primer adapter molecules which allow improved isolation and production of such nucleic acid molecules, increasing both product recovery and speed of isolation.

### BACKGROUND OF THE INVENTION

### cDNA and cDNA Libraries

In examining the structure and physiology of an organism, tissue or cell, it is often desirable to determine its genetic content. The genetic framework of an organism is encoded in the double-stranded sequence of nucleotide bases in the deoxyribonucleic acid (DNA) which is contained in the somatic and germ cells of the organism. The genetic content of a particular segment of DNA, or gene, is only manifested upon production of the protein which the gene encodes. In order to produce a protein, a complementary copy of one strand of the DNA double helix (the “coding” strand) is produced by polymerase enzymes, resulting in a specific sequence of ribonucleic acid (RNA). This particular type of RNA, since it contains the genetic message from the DNA for production of a protein, is called messenger RNA (mRNA).

Within a given cell, tissue or organism, there exist myriad mRNA species, each encoding a separate and specific protein. This fact provides a powerful tool to investigators interested in studying genetic expression in a tissue or cell—mRNA molecules may be isolated and further manipulated by various molecular biological techniques, thereby allowing the elucidation of the full functional genetic content of a cell, tissue or organism.

One common approach to the study of gene expression is the production of complementary DNA (cDNA) clones. In this technique, the mRNA molecules from an organism are isolated from an extract of the cells or tissues of the organism. This isolation often employs solid chromatography matrices, such as cellulose or Sepharose, to which oligomers of thymidine (T) have been complexed. Since the 3′ termini on all eukaryotic mRNA molecules contain a string of adenosine (A) bases, and since A binds to T, the mRNA molecules can be rapidly purified from other molecules and substances in the tissue or cell extract. From these purified mRNA molecules, cDNA copies may be made using an enzyme having reverse transcriptase (RT) activity, which results in the production of single-stranded cDNA molecules complementary to all or a portion of the mRNA templates. Incubating the single-stranded cDNA under appropriate conditions allows synthesis of double-stranded DNA which may then be inserted into a plasmid or a vector.

This entire process, from isolation of mRNA to insertion of the cDNA into a plasmid or vector to growth of host cell populations containing the isolated gene, is termed “cDNA cloning.” If cDNAs are prepared from a number of different mRNAs, the resulting set of cDNAs is called a “cDNA library,” an appropriate term since the set of cDNAs represents the different populations of functional genetic information (genes) present in the source cell, tissue or organism. Genotypic analysis of these cDNA libraries can yield much information on the structure and function of the organisms from which they were derived.

In traditional production methods, the cDNA molecules must be size fractionated and multiple phenol/chloroform extractions and ethanol precipitations performed. Each of these requirements has inherent disadvantages, such as product loss and limitations in cDNA yield due to multiple extractions/precipitations (Lambert, K. N., and Williamson, V. M., Nucl. Acids Res. 21(3):775-776 (1993)).

These disadvantages have been partially addressed in the literature. For example, several investigators have reported methods for the isolation of polyA+ mRNA from cell and tissue samples by binding the mRNA to latex or paramagnetic beads coupled with oligo(dT); single-stranded cDNA molecules may then be produced by reverse transcription of these immobilized mRNA molecules (Lambert, K. N., and Williamson, V. M., Nucl. Acids Res. 21(3):775-776 (1993); Kuribayashi-Ohta, K., et al., Biochim. Biophys. Acta 1156:204-212 (1993); Sasaki, Y. F., et al., Nucl. Acids Res. 22(6):987-992 (1994); Mészáros, M., and Morton, D. B., BioTechniques 20(3):413-419 (1996); Fellman, F., et al., BioTechniques 21(5):766-770 (1996)). Such solid phase synthesis methods are less prone to the yield limitations resulting from the extraction/precipitation steps of the traditional methods.

However, these methods still have several important limitations. For example, each of these methods relies on PCR amplification prior to cloning of the cDNA molecules, often resulting in biased cDNA libraries (i.e., highly expressed sequences predominate over those that are expressed in lower quantities). In addition, these methods often are less efficient than conventional cDNA synthesis methods which use solution hybridization of the primer-adapter to the template (i.e., rotational diffusion is required for increased hybridization rates; see Schmitz, K. S., and Schurr, J. M., J. Phys. Chem. 76:534-545 (1972); Ness, J. V., and Hahn, W. E., Nucl. Acids Res. 10(24):8061-8077 (1982)). Finally, the above-described techniques use heat or chemical denaturation to release the nascent cDNA molecules from the solid phase for further processing, which can result in product loss and/or damage.

Thus, a need exists in the art for methods that provide for rapid, high yield synthesis, isolation and manipulation of nucleic acid molecules from small quantities of RNA (total RNA or poly A+ mRNA). The present invention provides such methods.

### SUMMARY OF THE INVENTION

The present invention is directed to methods useful for the production and isolation of nucleic acid molecules (single- and double-stranded) from small amounts of input nucleic acid molecules. More particularly, the invention provides methods for the production of a cDNA molecule (single- or double-stranded) from an RNA template (e.g., single-stranded mRNA or polyA+ RNA) by using ligand-coupled primer-adapter molecules. Such primer-adapter molecules may also be used in accordance with the invention to isolate mRNA or polyA+ RNA molecules from an RNA-containing sample.

Specifically, the invention is directed to a method for producing a nucleic acid molecule comprising mixing a nucleic acid template, preferably a mRNA or a polyA+ RNA molecule, with a polypeptide having polymerase and/or reverse transcriptase activity and a primer-adapter nucleic acid molecule, wherein the primer-adapter nucleic acid molecule comprises one or more ligand molecules and one or more cleavage sites (preferably a restriction endonuclease cleavage site or an endonuclease cleavage site). This primer-adapter may be designed to hybridize to any portion of the template. Upon incubation under appropriate conditions, a first nucleic acid molecule (e.g., a single-stranded cDNA) complementary to all or a portion of the template is made. This first nucleic acid molecule contains the primer-adapter (preferably at or near its termini) which facilitates isolation of the first nucleic acid molecule and/or any nucleic acid molecule hybridized to the first nucleic acid molecule. Thus, if the first nucleic acid molecule (e.g., single-stranded cDNA) serves as a template to make a second nucleic acid molecule (e.g., forming a double-stranded molecule such as a double-stranded cDNA), the double-stranded molecule can be isolated using the primer-adapter contained in the molecule. Likewise, the template-first nucleic acid hybrid formed during synthesis of the first nucleic acid molecule can be isolated. If desired, the primer-adapter may be included at any step or at multiple steps during nucleic acid synthesis. For example, primer-adapter molecules may be added during the first, second, third, fourth, etc., synthesis step (the first synthesis step making a nucleic acid molecule complementary to all or a portion of the template) or can be added in multiple or all such synthesis steps. Multiple synthesis with primer-adapter molecules may result in a synthesized nucleic acid molecule having more than one primer-adapter.

To isolate mRNA or polyA+ RNA from RNA-containing samples, one or more mRNA- or polyA+ RNA-specific primer-adapters is used. Such a primer-adapter is hybridized to the mRNA and/or polyA+ RNA to form a primer-adapter/polyA+ RNA hybrid. The primer-adapter can then facilitate isolation of the mRNA and/or polyA+ RNA from a sample. In this aspect of the invention, since the primer-adapter is hybridized to the molecule of interest and can be removed by denaturation, cleavage sites in the primer-adapter are not needed.

The primer-adapter molecules of the invention may also be used to isolate specific nucleic acid sequences. By using one or more target-specific primer-adapters capable of hybridizing to one or more sequences of interest, the invention allows selection and isolation of specific nucleic acid molecules (e.g., genes or portions thereof) from a population of nucleic acid molecules. In accordance with the invention, the use of two or more such target-specific primer-adapters (each directed to a different sequence) allows selection of more than one different sequence of interest. Alternatively, two or more target-specific primer-adapters directed to different portions of a sequence of interest facilitates selection of such sequences by reducing background contamination. Because, in this aspect of the invention, the target-specific primer-adapter hybridizes to the desired molecule and can be removed by denaturation, cleavage sites in the target-specific primer-adapter are not needed.

In accordance with the invention, the primer-adapter molecules facilitate isolation of molecules comprising such primer-adapters by relying on the ligand portion of the primer-adapter. After the primer-adapter is bound (hybridized or incorporated during synthesis) to the nucleic acid molecule, the ligand portion of the primer-adapter allows selective isolation of the molecule containing the primer-adapter. Such isolation may be accomplished by ligand-hapten interactions, where the hapten is bound to, for example, a solid support. Once bound to the solid support, the molecules of interest (primer-adapter containing nucleic acid molecules) can be separated from contaminating nucleic acids and proteins by washing the support matrix with a solution, preferably a buffer or water. Cleavage of one or more of the cleavage sites within the primer-adapter then allows for removal of the nucleic acid molecule of interest from the solid support, leaving the ligand bound to the hapten of the solid support. Alternatively, where the primer-adapter is hybridized to the nucleic acid molecule of interest, isolation can be accomplished by denaturation of the primer-adapter from the desired molecules and/or by cleavage of the cleavage sites within the primer-adapter molecule.

Preferred solid supports for use in the invention include, but are not limited to, nitrocellulose, diazocellulose, glass, polystyrene, polyvinylchloride, polypropylene, polyethylene, dextran, Sepharose, agar, starch, nylon, latex beads, magnetic beads, paramagnetic beads, superparamagnetic beads or microtitre plates and most preferably a magnetic bead, a paramagnetic bead or a superparamagnetic bead, that comprises one or more hapten molecules specifically recognizing and binding to the ligand molecule.

Particularly preferred hapten molecules according to this aspect of the invention include without limitation: (i) avidin and streptavidin; (ii) protein A, protein G, a cell-surface Fc receptor or an antibody-specific antigen; (iii) an enzyme-specific substrate; (iv) polymyxin B or endotoxin-neutralizing protein (ENP); (v) Fe⁺⁺⁺; (vi) a transferrin receptor; (vii) an insulin receptor; (viii) a cytokine (e.g., growth factor, interleukin or colony-stimulating factor) receptor; (ix) CD4; (x) spectrin or fodrin; (xi) ICAM-1 or ICAM-2; (xii) C3bi, fibrinogen or Factor X; (xii) ankyrin; (xiv) integrins α₁β₁, α₂β₁, α₃β₁, α₆β₁, α₇β₁ and α₆β₅; (xv) integrins α₁β₁, α₂β₁, α₃β₁ and αᵥβ₃; (xvi) integrins α₃β₁, α₄β₁, α₄β₇, α₅β₅; αᵥβ₁, α_{IIb}β₃, αᵥβ₃ and αᵥβ₆; (xvii) integrins αᵥβ₁ and αᵥβ₃; (xviii) vitronectin; (xix) fibronectin; (xx) collagen; (xxi) laminin; (xxii) glycophorin; Mac-1; (xxiv) LFA-1; (xxv) β-actin; (xxvi) gp120; (xxvii) cytokines (growth factors, interleukins or colony-stimulating factors); (xxviii) insulin; (xxix) ferrotransferrin; (xxx) apotransferrin; (xxxi) lipopolysaccharide; (xxxii) an enzyme; (xxxiii) an antibody; and (xxxiv) biotin.

Particularly preferred ligand molecules for use according to the invention, which correspond in order to the above-described hapten molecules, include without limitation: (i) biotin; (ii) an antibody; (iii) an enzyme; (iv) lipopolysaccharide; (v) apotransferrin; (vi) ferrotransferrin; (vii) insulin; (viii) cytokines (growth factors, interleukins or colony-stimulating factors); (ix) gp120; (x) β-actin; (xi) LFA-1; (xii) Mac-1; (xiii) glycophorin; (xiv) laminin; (xv) collagen; (xvi) fibronectin; (xvii) vitronectin; (xviii) integrins αᵥβ₁ and αᵥβ₃; (xix) integrins α₃β₁, α₄β₁, α₄β₇, α₅β₁, αᵥβ₁, α_{IIb}β₃, αᵥβ₃ and αᵥβ₆; (xx) integrins α₁β₁, α₂β₁, α₃β₁ and αᵥβ₃; (xxi) integrins α₁β₁, α₂β₁, α₃β₁, α₆β₁, α₇β₁ and α₆β₅; (xxii) ankyrin; (xxiii) C3bi, fibrinogen or Factor X; (xxiv) ICAM-1 or ICAM-2; (xxv) spectrin or fodrin; (xxvi) CD4; (xxvii) a cytokine (e.g., growth factor, interleukin or colony-stimulating factor) receptor; (xxviii) an insulin receptor; (xxix) a transferrin receptor; (xxx) Fe⁺⁺⁺; (xxxi) polymyxin B or endotoxin-neutralizing protein (ENP); (xxxii) an enzyme-specific substrate; (xxxiii) protein A, protein G, a cell-surface Fc receptor or an antibody-specific antigen; and (xxxiv) avidin and streptavidin.

The invention thus relates to a method for making a nucleic acid molecule comprising

(a) mixing a polypeptide having polymerase and/or reverse transcriptase activity with a nucleic acid template and a primer-adapter of the invention; and

(b) incubating the mixture under conditions sufficient to make a first nucleic acid molecule which comprises the primer-adapter (preferably at or near its 5′ or 3′ termini) and which is complementary to all or a portion of the template. [If a DNA polymerase is used in accordance with the invention, the primer-adapter may be located at or near the 3′ terminus, while if a reverse transcriptase is used the primer-adapter may be located at or near the 5′ terminus of the synthesized nucleic acid molecule. In accordance with the invention, the first nucleic acid molecule may be used as a template to make a second nucleic acid molecule complementary to all or a portion of the first nucleic acid molecule. If a primer-adapter is used in this synthesis, a double-stranded nucleic acid molecule is produced which comprises a primer-adapter at or near each terminus, although on different strands of the molecule. However, the primer-adapter may be omitted from this second synthesis thereby providing for a double-stranded nucleic acid molecule having a primer-adapter at one terminus.

If desired, the primer-adapters of the invention may be used in methods for amplifying a nucleic acid molecule. Such methods comprise

(a) contacting a polypeptide having polymerase and/or reverse transcriptase activity with a nucleic acid template and two or more primer-adapters; and

(b) incubating the mixture under conditions sufficient to amplify a nucleic acid molecule complementary to all or a portion of the template.

Such amplification methods may specifically comprise

(a) contacting a double-stranded nucleic acid molecule to be amplified with a polypeptide having polymerase and/or reverse transcriptase activity, a first primer-adapter complementary to a portion of the first strand of the double-stranded molecule and a second primer-adapter complementary to a portion of the second strand of the double-stranded molecule;

(b) incubating the mixture under conditions sufficient to make a third strand nucleic acid molecule comprising the first primer-adapter and which is complementary to all or a portion of the first strand, and a fourth strand nucleic acid molecule comprising the second primer-adapter and which is complementary to all or a portion of the second strand;

(c) denaturing the second and fourth, and the first and third, strands to form single-stranded nucleic acid molecules; and

(d) repeating steps (a)-(c) one or more times.

In this aspect of the invention, the first primer-adapter or the second primer-adapter may be replaced with any oligonucleotide primer to prime synthesis of a nucleic acid molecule.

In a preferred aspect of the invention, RNA (e.g., mRNA or polyA+ RNA) is used as a template for DNA synthesis. This preferred method comprises mixing the RNA template with one or more polypeptides having reverse transcriptase activity and a primer and incubating the mixture under conditions sufficient to make a DNA (e.g., a cDNA) molecule complementary to all or a portion of the RNA template. The synthesized DNA molecule may then be used as a template for additional DNA synthesis or DNA amplification. In accordance with this aspect of the invention, a cDNA library may be produced when using a population of RNA molecules (for example, RNA isolated from a cell or tissue).

For isolating mRNA or polyA+ RNA in accordance with the invention, the method may specifically comprise:

(a) obtaining a sample containing (or thought to contain) mRNA and/or polyA+ RNA;

(b) contacting the sample with one or more primer-adapters capable of selectively binding to mRNA and/or polyA+ RNA; and

(c) isolating the mRNA and/or polyA+ RNA from the sample.

For isolating specific or desired nucleic acid molecules, the invention may specifically comprise:

(a) obtaining a sample containing (or thought to contain) one or more desired nucleic acid molecules;

(b) contacting the sample with one or more primer-adapters capable of selectively binding to one or more of the desired nucleic acid molecules; and

(c) isolating the desired nucleic acid molecules from the sample.

In a preferred aspect, the sample containing the desired molecules is a population of double-stranded or single-stranded cDNA molecules. Thus, the invention relates to a method of isolating one or more desired nucleic acid molecules comprising:

(a) obtaining a sample containing a population of cDNA molecules which contain (or are thought to contain) one or more desired cDNA molecules;

(b) contacting the sample with one or more target-specific primer-adapters capable of specifically binding to one or more of the desired cDNA molecules; and

(c) isolating the desired cDNA molecules from the sample.

In accordance with the invention, the target-specific primer-adapters may be used in selection of a specific cDNA molecule after the cDNA molecule is synthesized from the RNA template (binding to the RNA/cDNA double-stranded molecule or binding to the single-stranded cDNA molecule after removing the RNA strand). Alternatively, the target-specific primer-adapters may be used to bind the double-stranded cDNA molecule. Such target-specific primer-adapters may also be used in accordance with the invention to select one or more desired molecules from a population of amplified nucleic acid molecules.

The invention is also directed to vectors, including expression vectors, comprising the cDNA molecules or nucleic acid molecules produced in accordance with the invention, and to host cells comprising these cDNA molecules, nucleic acid molecules or vectors. The invention also provides methods for producing a recombinant polypeptide comprising culturing these host cells under conditions favoring the expression of a recombinant polypeptide and isolating the polypeptide, and provides recombinant polypeptides produced according to these methods.

In other preferred aspects, the invention is directed to kits for the production of a nucleic acid molecule or a cDNA molecule comprising a carrier means such as a box, carton, or the like being compartmentalized to receive in close confinement therein one or more containers, such as tubes, vials, bottles, ampules and the like, wherein a first container comprises a primer-adapter molecule comprising one or more ligand molecules, preferably biotin, and which comprises one or more cleavage sites, preferably one or more restriction endonuclease cleavage sites. The invention is also directed to such kits comprising additional containers which may contain one or more polypeptides having reverse transcriptase activity and/or polymerase activity. According to the invention, more than one polypeptide may be included in the same or different containers. The invention is also directed to kits comprising additional containers which may contain a solid support having one or more haptens capable of specifically binding the ligand or ligands of the primer-adapters of the invention. The invention is also directed to kits comprising additional containers which may contain one or more endonucleases which recognize and cleave the cleavage sites in the primer-adapters of the invention.

Other preferred embodiments of the present invention will be apparent to one of ordinary skill in light of the following drawings and description of the invention, and of the claims.

### BRIEF DESCRIPTION OF THE FIGURES

FIG. 1 is a depiction of the production and isolation of a double-stranded cDNA molecule and its ligation into a plasmid vector (pCMVSPORT), according to the methods of the present invention. “B” denotes biotin molecules (and thus sites of biotinylation of the cDNA molecule), and “RE” denotes location of restriction endonuclease cleavage sites used to facilitate removal of the cDNA from the solid phase support following isolation.

### DETAILED DESCRIPTION OF THE INVENTION

The present invention is particularly suited for the rapid production and isolation of cDNA libraries from small amounts of poly A+ RNA or mRNA in a high-throughout manner. In a preferred aspect of the invention, a population of single-stranded poly A+ RNA or mRNA is hybridized in solution with a ligand-coupled primer adapter (non-specific or gene-specific). As used herein, the term “primer-adapter” refers to a nucleic acid molecule which is capable of specifically binding (e.g., hybridizing) to a template nucleic acid molecule (e.g., a mRNA or polyA+ RNA molecule). In a particularly preferred embodiment of the invention, the primer-adapter allows priming of the transcription, reverse transcription, polymerization or elongation of a nucleic acid molecule complementary to all or a portion of the template nucleic acid molecule.

According to the invention, the first and second strand cDNA reactions are preferably performed in one tube, introducing the ligand at or near the 3′ end of the double-stranded cDNA produced. The ligand-coupled cDNA may then be isolated by binding to a solid support coupled with a hapten to which the cDNA will bind through ligand-hapten interactions, thereby allowing the concentration of the cDNA and exchange of the buffer without organic extraction and precipitation. Subsequently, the bound cDNA is released from the solid phase support by restriction enzyme digestion. This asymmetric cDNA is then cloned directionally into a vector that contains the appropriate termini (one terminus matches the restriction site used to release the cDNA and the other terminus is blunt ended). Subsequent or prior to cloning into a vector, specific cDNA sequences (e.g., genes or gene fragments) may be selectively isolated using target-specific primer-adapters of the invention. In addition to the elimination of multiple time-consuming extractions and precipitations, the methods of the invention eliminate the need for DNA adapters and cDNA fractionation (normally a necessary step to remove excess unligated adapters). The invention thus facilitates rapid production and isolation of larger amounts of cDNA and the construction of cDNA libraries from nanogram amounts of poly A+ RNA or mRNA without the need for PCR amplification. The invention also provides a simple selection technique which allows isolation of desired genes or gene fragments from the constructed cDNA library.

### Sources of Nucleic Acid Template Molecules

Using the methods of the invention, nucleic acid molecules and in particular cDNA molecules may be prepared from a variety of nucleic acid template molecules. Preferred nucleic acid molecules for use in the present invention include single-stranded or double-stranded RNA. More preferred nucleic acid molecules include polyadenylated RNA (polyA+ RNA), messenger RNA (mRNA), transfer RNA (tRNA) and ribosomal RNA (rRNA) molecules, and most preferred are mRNA and polyA+ RNA molecules.

The nucleic acid template molecules that are used to prepare nucleic acid or cDNA molecules according to the methods of the present invention may be prepared synthetically according to standard organic chemical synthesis methods that will be familiar to one of ordinary skill. More preferably, the nucleic acid template molecules may be obtained from natural sources, such as a variety of cells, tissues, organs or organisms. Cells that may be used as sources of nucleic acid molecules may be prokaryotic (bacterial cells, including those of species of the genera Escherichia, Bacillus, Serratia, Salmonella, Staphylococcus, Streptococcus, Clostridium, Chlamydia, Neisseria, Treponema, Mycoplasma, Borrelia, Legionella, Pseudomonas, Mycobacterium, Helicobacter, Erwinia, Agrobacterium, Rhizobium, and Streptomyces) or eukaryotic (including fungi (especially yeasts), plants, protozoans and other parasites, and animals including insects (particularly Drosophila spp. cells), nematodes (particularly Caenorhabditis elegans cells), and mammals (particularly human cells)).

Mammalian somatic cells that may be used as sources of nucleic acids include blood cells (reticulocytes and leukocytes), endothelial cells, epithelial cells, neuronal cells (from the central or peripheral nervous systems), muscle cells (including myocytes and myoblasts from skeletal, smooth or cardiac muscle), connective tissue cells (including fibroblasts, adipocytes, chondrocytes, chondroblasts, osteocytes and osteoblasts) and other stromal cells (e.g., macrophages, dendritic cells, Schwann cells). Mammalian germ cells (spermatocytes and oocytes) may also be used as sources of nucleic acids for use in the invention, as may the progenitors, precursors and stem cells that give rise to the above somatic and germ cells. Also suitable for use as nucleic acid sources are mammalian tissues or organs such as those derived from brain, kidney, liver, pancreas, blood, bone marrow, muscle, nervous, skin, genitourinary, circulatory, lymphoid, gastrointestinal and connective tissue sources, as well as those derived from a mammalian (including human) embryo or fetus.

Any of the above prokaryotic or eukaryotic cells, tissues and organs may be normal, diseased, transformed, established, progenitors, precursors, fetal or embryonic. Diseased cells may, for example, include those involved in infectious diseases (caused by bacteria, fungi or yeast, viruses (including AIDS) or parasites), in genetic or biochemical pathologies (e.g., cystic fibrosis, hemophilia, Alzheimer's disease, muscular dystrophy or multiple sclerosis) or in cancerous processes. Transformed or established animal cell lines may include, for example, COS cells, CHO cells, VERO cells, BHK cells, HeLa cells, HepG2 cells, K562 cells, F9 cells and the like. Other cells, cell lines, tissues, organs and organisms suitable as sources of nucleic acids for use in the present invention will be apparent to one of ordinary skill in the art.

Once the starting cells, tissues, organs or other samples are obtained, nucleic acid molecules (such as mRNA) may be isolated therefrom by methods that are well-known in the art (See, e.g., Maniatis, T., et al., Cell 15:687-701 (1978); Okayama, H., and Berg, P., Mol. Cell. Biol. 2:161-170 (1982); Gubler, U., and Hoffman, B. J., Gene 25:263-269 (1983)). As discussed, the invention provides an improvement in isolating mRNA and/or polyA+ RNA from samples. The use of the primer-adapters of the invention, which specifically recognize and bind polyA+ RNA or mRNA, allows for such selection. Preferably, the primer-adapter recognizes and hybridizes to the polyA tail of the mRNA or polyA+ RNA. Such primer-adapters may include an primer-adapters comprising oligo(dT). Once bound, use of the ligand portion of the primer-adapter allows isolation of the desired RNA molecule. The polyA+ RNA or mRNA molecules thus isolated may then be used to prepare cDNA molecules and cDNA libraries using the methods of the present invention.

### Synthesis of Nucleic Acid Molecules

In the practice of the invention, nucleic acid molecules and in particular cDNA molecules or cDNA libraries comprising one or more ligand molecules are produced by mixing a nucleic acid template obtained as described above, which is preferably a mRNA molecule or a polyA+ RNA molecule, with one or more polypeptides having polymerase activity and/or reverse transcriptase activity and with a one or more primer-adapters of the invention. Under conditions favoring the reverse transcription and/or polymerization of the input nucleic acid molecule, synthesis of a nucleic acid molecule complementary to all or a portion of the template is accomplished. Preferred polypeptides (e.g., enzymes) having reverse transcriptase and/or polymerase activity to be used in the present invention include, but are not limited to, Moloney Murine Leukemia Virus (M-MLV) reverse transcriptase, Rous Sarcoma Virus (RSV) reverse transcriptase, Avian Myeloblastosis Virus (AMV) reverse transcriptase, Rous Associated Virus (RAV) reverse transcriptase, Myeloblastosis Associated Virus (MAV) reverse transcriptase, Human Immunodeficiency Virus (HIV) reverse transcriptase, retroviral reverse transcriptase, retrotransposon reverse transcriptase, hepatitis B reverse transcriptase, cauliflower mosaic virus reverse transcriptase, bacterial reverse transcriptase, Thermus thermophilus (Tth) DNA polymerase, Thermus aquaticus (Taq) DNA polymerase, Thermotoga neopolitana (Tne) DNA polymerase, Thermotoga maritima (Tma) DNA polymerase, Thermococcus litoralis (Tli or VENT™) DNA polymerase, Pyrococcus furiosus (Pfu or DEEPVENT™) DNA polymerase, Pyrococcus woosii (Pwo) DNA polymerase, Bacillus sterothermophilus (Bst) DNA polymerase, Sulfolobus acidocaldarius (Sac) DNA polymerase, Thermoplasma acidophilum (Tac) DNA polymerase, Thermus flavus (Tfl/Tub) DNA polymerase, Thermus ruber (Tru) DNA polymerase, Thermus brockianus (DYNAZYME™) DNA polymerase, Methanobacterium thermoautotrophicum (Mth) DNA polymerase, and mutants, variants and derivatives thereof. Particularly preferred for use in the invention are the variants of these enzymes that are substantially reduced in RNase H activity. By an enzyme “substantially reduced in RNase H activity” is meant that the enzyme has less than about 20%, more preferably less than about 15%, 10% or 5%, and most preferably less than about 2%, of the RNase H activity of a wildtype or “RNase H⁺” enzyme such as wildtype M-MLV or AMV reverse transcriptases. The RNase H activity of any enzyme may be determined by a variety of assays, such as those described, for example, in U.S. Pat. No. 5,244,797, in Kotewicz, M. L., et al., Nucl. Acids Res. 16:265 (1988) and in Gerard, G. F., et al., FOCUS 14(5):91 (1992), the disclosures of all of which are fully incorporated herein by reference.

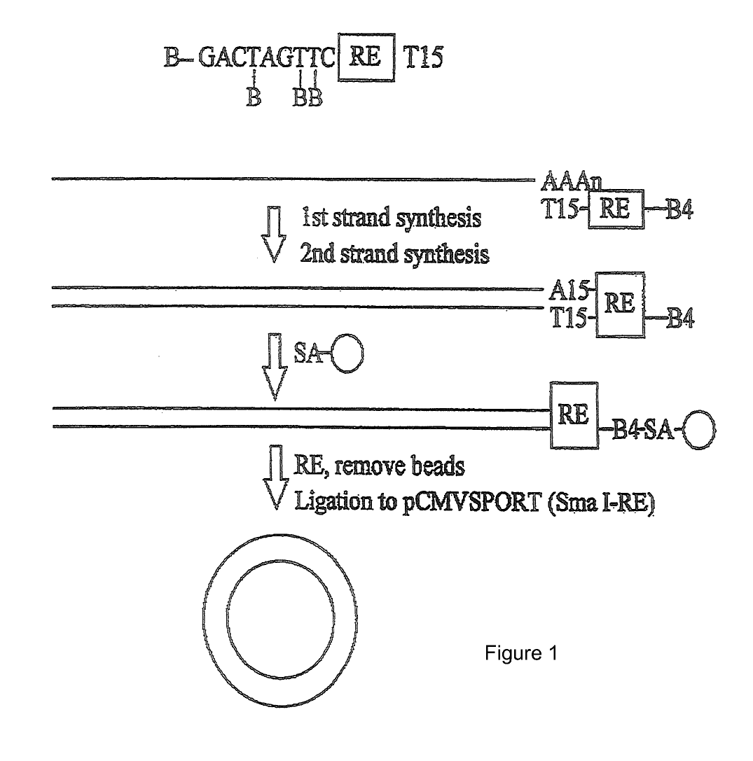

Any ligand to which a hapten molecule will bind may be used to form the ligand-coupled primer-adapter molecule used in the present methods. Suitable ligands for this purpose include, but are not limited to: (i) biotin; (ii) an antibody; (iii) an enzyme; (iv) lipopolysaccharide; (v) apotransferrin; (vi) ferrotransferrin; (vii) insulin; (viii) cytokines (growth factors, interleukins or colony-stimulating factors); (ix) gp120; (x) β-actin; (xi) LFA-1; (xii) Mac-1; (xiii) glycophorin; (xiv) laminin; (xv) collagen; (xvi) fibronectin; (xvii) vitronectin; (xviii) integrins αᵥβ₁ and αᵥβ₃; (xix) integrins α₃β₁, α₄β₁, α₄β₇, α₅β₁, αᵥβ₁, α_{IIb}β₃, αᵥβ₃ and αᵥβ₆; (xx) integrins α₁β₁, α₂β₁, α₃β₁ and αᵥβ₃; (xxi) integrins α₁β₁, α₂β₁, α₃β₁, α₆β₁, α₇β₁ and α₆β₅; (xxii) ankyrin; (xxiii) C3bi, fibrinogen or Factor X; (xxiv) ICAM-1 or ICAM-2; (xxv) spectrin or fodrin; (xxvi) CD4; (xxvii) a cytokine (e.g., growth factor, interleukin or colony-stimulating factor) receptor; (xxviii) an insulin receptor; (xxix) a transferrin receptor; (xxx) Fe⁺⁺⁺; (xxxi) polymyxin B or endotoxin-neutralizing protein (ENP); (xxxii) an enzyme-specific substrate; (xxxiii) protein A, protein G, a cell-surface Fc receptor or an antibody-specific antigen; and (xxxiv) avidin and streptavidin. Most preferred for use in the methods of the invention is biotin. The ligand-coupled primer-adapter nucleic acid molecules, in which one or more ligand molecules are attached (preferably covalently) to one or more nucleotides of the primer-adapter molecule (see, for example, FIG. 1), may be produced using conventional organic synthesis methods that are familiar to one of ordinary skill in the art. For example, the oligonucleotide may be biotinylated at the 5′ terminus by first producing 5′ amino (NH₂) groups followed by Cab-NHS ester addition (Langer, P. R., et al., Proc. Natl. Acad. Sci. USA 78:6633 (1981)). In a particularly preferred aspect of the invention, a primer-adapter molecule comprising one or more, two or more; three or more or four or more ligand molecules, most preferably biotin molecules, is prepared.

In addition to the ligand molecules, the primer-adapter molecule also preferably comprises one or more endonuclease cleavage sites, preferably restriction endonuclease cleavage sites. These sites facilitate the release of the newly synthesized nucleic acid molecule comprising the primer-adapter from the hapten-coupled solid support. Examples of endonucleases which can be used in accordance with the invention include, but are not limited to, GeneII. Examples of restriction endonucleases which can be used in accordance with the invention include, but are not limited to, AluI, Eco47 III, EcoRV, FspI, HpaI, MscI, NruI, PvuII, RsaI, ScaI, SmaI, SspI, StuI, ThaI, AvaI, BamHI, BanII, BglII, ClaI, EcoRI, HindIII, HpaII, KpnI, MseI, NcoI, NdeI, NotI, PstI, PvuI, SacI/SstI, SalI, XbaI, XhoI and I-CeuI.

The restriction endonuclease sites engineered into the primer-adapter molecule are preferably chosen to result in either blunt ends or sticky ends. Examples of blunt-end restriction enzymes, the recognition sites for which may be engineered into the primer-adapter molecules of the invention, include without limitation AluI, Eco47 III, EcoRV, FspI, HpaI, MscI, NruI, PvuII, RsaI, ScaI, SmaI, SspI, StuI and ThaI.

Examples of sticky-end restriction enzymes, the recognition sites for which may be engineered into the primer-adapter molecules of the invention, include without limitation AvaI, BamHI, BanII, BglII, ClaI, EcoRI, HindIII, HpaII, KpnI, MseI, NcoI, NdeI, NotI, PstI, PvuI, SacI/SstI, SalI, Xba, XhoI and I-CeuI.

In a particularly preferred aspect of the invention, the primer-adapter molecule is engineered to contain a site recognized by rare cutting restriction endonucleases, for example, those recognizing 8 or more bases (e.g., a 8-basepair cutter, etc.). Such restriction sites may include a NotI restriction site, a I-CeuI restriction site, a PI-PspI restriction site, an I-PpoI restriction site, a PI-TliI restriction site and a PI-FceI restriction site. The above-mentioned restriction enzymes, and others that may be equivalently used in the methods of the present invention, are available commercially, for example from Life Technologies, Inc. (Rockville, Md.). See also Roberts, R. J., Nucl. Acids Res. 17(Suppl.):r347-r387 (1989), for other examples of restriction enzymes and their cleavage sites.

Once the ligand-coupled primer-adapter molecule has been obtained, it is used to produce nucleic acid molecules from the input nucleic acid using any of a number of well-known techniques. Such synthetic techniques involve hybridization of the primer-adapter to the nucleic acid template and extending the primer-adapter to make a nucleic acid molecule complementary to all or a portion of the template. Such synthesis is accomplished in the presence of nucleotides (e.g., deoxyribonucleoside triphosphates (dNTPs), dideoxyribonucleoside triphosphates (ddNTPs) or derivatives thereof) and one or more polypeptides having polymerase and/or reverse transcriptase activity. The primer-adapters of the invention may be used in any nucleic acid synthesis reaction including cDNA synthesis, nucleic acid amplification and nucleic acid sequencing, using well-known techniques. For synthesis of cDNA, the primer-adapter molecules of the invention may be used in conjunction with methods of cDNA synthesis such as those described in Example 1 below, or others that are well-known in the art (see, e.g., Gubler, U., and Hoffman, B. J., Gene 25:263-269 (1983); Krug, M. S., and Berger, S. L., Meth. Enzymol. 152:316-325 (1987); Sambrook, J., et al., Molecular Cloning: A Laboratory Manual, 2nd ed., Cold Spring Harbor, N.Y.: Cold Spring Harbor Laboratory Press, pp. 8.60-8.63 (1987)), to produce cDNA molecules or libraries.

Alternatively, the primer adapter molecules of the invention may be used in single-tube synthesis of cDNA molecules according to the invention. In this approach, the input nucleic acid molecule (preferably a mRNA or polyA+ RNA molecule) is hybridized in solution with the primer-adapter molecule of the invention, and the hybridized complex is contacted with a polypeptide (e.g., an enzyme) having reverse transcriptase activity (which is preferably any of those described above) in the presence of dNTPs and cofactors needed for cDNA synthesis. Following first strand synthesis, the second cDNA strand may then be synthesized in the same reaction vessel by a modified Gubler-Hoffman reaction (D'Alessio, J. M., et al., Focus 9:1 (1987)). Other techniques of cDNA synthesis in which the methods of the invention may be advantageously used will be readily apparent to one of ordinary skill in the art.

### Isolation of Nucleic Acid Molecules

According to the present methods, single-stranded or double-stranded nucleic acid molecules (e.g., cDNA molecules or cDNA libraries) comprising one or more primer-adapters will be produced. Such nucleic acid molecules or libraries may then be rapidly isolated from solution by binding the nucleic acid molecules to a solid support comprising one or more hapten molecules that will bind the ligands.

In the practice of the invention, any solid support to which a ligand-specific hapten molecule can be bound may be used. Preferred such solid phase supports include, but are not limited to, nitrocellulose, diazocellulose, glass, polystyrene, polyvinylchloride, polypropylene, polyethylene, dextran, Sepharose, agar, starch, nylon, beads and microtitre plates. Preferred are beads made of glass, latex or a magnetic material, and particularly preferred are magnetic, paramagnetic or superparamagnetic beads. Linkage of the hapten molecule to the solid support can be accomplished by any method of hapten coupling such as covalent, hydrophobic or ionic coupling (including coating) that will be familiar to one of ordinary skill in the art.

According to the invention, any hapten molecule having the capability of binding the ligand molecule that is coupled to the primer-adapter molecule (and that therefore is contained in the nucleic acid molecules produced by the present methods) may be used. Particularly preferred hapten molecules for use in the invention (which correspond in order to the ligand molecules listed above) include without limitation: (i) avidin and streptavidin; (u) protein A, protein G, a cell-surface Fc receptor or an antibody-specific antigen; (iii) an enzyme-specific substrate; (iv) polymyxin B or endotoxin-neutralizing protein (ENP); (v) Fe⁺⁺⁺; (vi) a transferrin receptor; (vii) an insulin receptor; (viii) a cytokine (e.g., growth factor, interleukin or colony-stimulating factor) receptor; (ix) CD4; (x) spectrin or fodrin; (xi) ICAM-1 or ICAM-2; (xii) C3bi, fibrinogen or Factor X; (xiii) ankyrin; (xiv) integrins α₁β₁, α₂β₁, α₃β₁, α₆β₁, α₇β₁ and α₆β₅; (xv) integrins α₁β₁, α₂β₁, α₃β₁ and αᵥβ₃; (xvi) integrins α₃β₁, α₄β₁, α₄β₇, α₅β₁, αᵥβ₁, α_{IIb}β₃, αᵥβ₃ and αᵥβ₆; (xvii) integrins αᵥβ₁ and αᵥβ₃; (xviii) vitronectin; (xix) fibronectin; (xx) collagen; (xxi) laminin; (xxii) glycophorin; Mac-1; (xxiv) LFA-1; (xxv) β-actin; (xxvi) gp120; (xxvii) cytokines (growth factors, interleukins or colony-stimulating factors); (xxviii) insulin; (xxix) ferrotransferrin; (xxx) apotransferrin; (xxxi) lipopolysaccharide; (xxxii) an enzyme; (xxxiii) an antibody; and (xxxiv) biotin.

For example, in a preferred aspect of the invention where the primer-adapter molecule and the newly synthesized nucleic acid molecules comprise biotin, a biotin-binding hapten such as avidin or streptavidin may be linked to the solid support. In a particularly preferred such aspect, the solid support used is avidin- or streptavidin-coupled magnetic, paramagnetic or superparamagnetic beads which are commercially available, for example, from Dynal A. S. (Oslo, Norway) or from Sigma (St. Louis, Mo.). Of course, the choice of hapten will depend upon the choice of ligand used in the production of the primer-adapter molecule; appropriate haptens for use in the methods of the invention will thus be familiar to one of ordinary skill in the art.

To isolate the nucleic acid molecules produced by the methods of the invention, the solution comprising the nucleic acid molecules which comprise the primer-adapters of the invention is contacted with the hapten-coupled solid support under conditions favoring binding of the ligand by the hapten. Typically, these conditions include incubation in a buffered salt solution, preferably a TRIS-, phosphate-, HEPES- or carbonate-buffered sodium chloride solution, more preferably a TRIS-buffered sodium chloride solution, still more preferably a solution comprising about 10-100 mM TRIS-HCl and about 300-2000 mM NaCl, and most preferably a solution comprising about 10 mM TRIS-HCl and about 1 M NaCl, at a pH of about 6-9, more preferably a pH of about 7-8, still more preferably a pH of about 7.2-7.6, and most preferably a pH of about 7.5. Incubation is preferably conducted at 0° C. to about 25° C., and most preferably at about 25° C., for about 30-120 minutes, preferably about 45-90 minutes, and most preferably about 60 minutes, to allow the binding of the ligand-coupled nucleic acid molecules to the hapten-coupled solid support.

Once the nucleic acid molecules have been bound to the solid phase support, unwanted or contaminant materials (such as buffers and enzymes from first and second strand synthesis reactions, untranscribed input RNA molecules, etc.) may be eliminated by simply removing them in the supernatants. For example, in a preferred aspect in which biotinylated cDNA molecules are bound to a avidin- or streptavidin-coupled solid phase, the contaminants may be removed by gently aspirating and discarding the supernatants. In a particularly preferred such aspect in which avidin- or streptavidin-coupled magnetic, paramagnetic or superparamagnetic beads are used as the solid support, the nucleic acid (e.g., cDNA)—containing beads are segregated from the supernatants using a magnet (such as a Magna-Sep Magnetic Particle Separator; Life Technologies, Inc.) and the supernatants are withdrawn using a pipette. Prior to their release from the solid support, the immobilized nucleic acid molecules are preferably washed one or more times, for example with one of the buffered salt solutions described above, to more fully remove unwanted materials.

Once the contaminants have been fully removed, the nucleic acid (e.g., cDNA) molecules may be released from the solid support by contacting the support with an endonuclease, which may be a restriction endonuclease, that specifically recognizes the sequence engineered into the primer-adapter molecule as described above, under conditions favoring the cleavage of the recognition sequence. In a particularly preferred such aspect of the invention in which a NotI and/or I-CeuI recognition sequence is engineered into the primer-adapter molecule (and is thus contained in the newly synthesized nucleic acid (e.g., cDNA) molecules), the solid support is contacted with a solution comprising NotI and/or I-CeuI. Of course, the choice of restriction enzyme used to release the nucleic acid molecules from the solid support will depend upon the specific recognition site engineered into the primer-adapter molecule and the possibility of that recognition site being present in the nucleic acid molecules. Preferred conditions for release of the nucleic acid molecules (e.g., cDNA or cDNA libraries) from the solid support include incubation at about 20° C. to about 40° C., preferably at about 25° C. to about 39° C., more preferably about 30° C. to about 37° C., and most preferably about 37° C., for about 30-180 minutes, preferably about 60-150 minutes, and most preferably about 120 minutes. Following their release from the solid support, the nucleic acid molecules (e.g., cDNA molecules or cDNA libraries) may be processed and further purified in accordance with the invention, or by techniques that are well-known in the literature (see, e.g., Gubler, U., and Hoffman, B. J., Gene 25:263-269 (1983); Krug, M. S., and Berger, S. L., Meth. Enzymol. 152:316-325 (1987); Sambrook, J., et al., Molecular Cloning: A Laboratory Manual, 2nd ed., Cold Spring Harbor, N.Y.: Cold Spring Harbor Laboratory Press, pp. 8.60-8.63 (1987)), and others that will be familiar to one of ordinary skill in the art.

### Kits

The present invention also provides kits for use in production and isolation of nucleic acid molecules (e.g., cDNA molecules or libraries). Kits according to this aspect of the invention comprise a carrier means, such as a box, carton, tube or the like, having in close confinement therein one or more containers, such as vials, tubes, ampules, bottles and the like, wherein a first container contains one or more primer-adapter nucleic acid molecules, which are preferably biotinylated primer-adapter nucleic acid molecules. In other aspects, the kits of the invention may further comprise one or more additional containers containing a hapten-coupled solid support, which may be any of the above-described solid supports and which is most preferably avidin- or streptavidin-coupled magnetic, paramagnetic or superparamagnetic beads. In additional aspects, the kits of the invention may further comprise one or more additional containers containing, for example, one or more nucleotides (e.g., dNTPs, ddNTPs or derivatives thereof) or one or more polypeptides (e.g., enzymes) having reverse transcriptase activity and/or polymerase activity, preferably any of those enzymes described above. Such nucleotides or derivatives thereof may include, but are not limited to, dUTP, dATP, dTTP, dCTP, dGTP, dITP, 7-deaza-dGTP, α-thio-dATP, α-thio-dTTP, α-thio-dGTP, α-thio-dCTP, ddUTP, ddATP, ddTTP, ddCTP, ddGTP, ddITP, 7-deaza-ddGTP, α-thio-ddATP, α-thio-ddTTP, α-thio-ddGTP, α-thio-ddCTP or derivatives thereof, all of which are available commercially from sources including Life Technologies, Inc. (Rockville, Md.), New England BioLabs (Beverly, Mass.) and Sigma Chemical Company (Saint Louis, Mo.). Additional kits according to the invention may comprise one or more additional containers containing one or more endonucleases or restriction enzymes used for release of the nucleic acid molecules (e.g., cDNA molecules or cDNA libraries) from the solid support. The kits encompassed by this aspect of the present invention may further comprise additional reagents (e.g., suitable buffers) and compounds necessary for carrying out nucleic acid reverse transcription and/or polymerization protocols.

### Uses

The present invention can be used in a variety of applications requiring rapid production and isolation of nucleic acid molecules. The invention is particularly suited for isolation of mRNA or polyA+ RNA molecules, for isolation of desired nucleic acid molecules from a population of nucleic acid molecules, and for production of nucleic acid molecules (particularly full-length cDNA molecules from small amounts of mRNA).

The invention is also directed to methods for the amplification of a nucleic acid molecule, and to nucleic acid molecules amplified by to these methods.

According to this aspect of the invention, a nucleic acid molecule may be amplified (i.e., additional copies of the nucleic acid molecule prepared) by amplifying the nucleic acid molecule (e.g., a cDNA molecules) of the invention according to any amplification method that is known in the art. Particularly preferred amplification methods according to this aspect of the invention include PCR (U.S. Pat. Nos. 4,683,195 and 4,683,202), Strand Displacement Amplification (SDA; U.S. Pat. No. 5,455,166; EP 0 684 315), and Nucleic Acid Sequence-Based Amplification (NASBA; U.S. Pat. No. 5,409,818; EP 0 329 822). Most preferred are those methods comprising one or more PCR amplifications.

The invention is also directed to methods that may be used to prepare recombinant vectors which comprise the nucleic acid molecules or amplified nucleic acid molecules of the present invention, to host cells which comprise these recombinant vectors, to methods for the production of a recombinant polypeptide using these vectors and host cells, and to recombinant polypeptides produced using these methods.

Recombinant vectors may be produced according to this aspect of the invention by inserting, using methods that are well-known in the art, one or more of the nucleic acid molecules or amplified nucleic acid molecules prepared according to the present methods into a vector (see FIG. 1). The vector used in this aspect of the invention may be, for example, a phage or a plasmid, and is preferably a plasmid. Preferred are vectors comprising cis-acting control regions to the nucleic acid encoding the polypeptide of interest. Appropriate trans-acting factors may be supplied by the host, supplied by a complementing vector or supplied by the vector itself upon introduction into the host.

In certain preferred embodiments in this regard, the vectors are expression vectors that provide for specific expression of the cDNA molecule or nucleic acid molecule of the invention, which vectors may be inducible and/or cell type-specific. Particularly preferred among such vectors are those inducible by environmental factors that are easy to manipulate, such as temperature and nutrient additives.

Expression vectors useful in the present invention include chromosomal-, episomal- and virus-derived vectors, e.g., vectors derived from bacterial plasmids or bacteriophages, and vectors derived from combinations thereof, such as cosmids and phagemids, and will preferably include at least one selectable marker such as a tetracycline or ampicillin resistance gene for culturing in a bacterial host cell. Prior to insertion into such an expression vector, the nucleic acid molecules (e.g., cDNA molecules) or amplified nucleic acid molecules of the invention should be operatively linked to an appropriate promoter, such as the phage lambda PL promoter, the E. coli lac, trp and tac promoters. Other suitable promoters will be known to the skilled artisan.

Among vectors preferred for use in the present invention include pQE70, pQE60 and pQE-9, available from Qiagen; pBS vectors, Phagescript vectors, Bluescript vectors, pNH8A, pNH16a, pNH18A, pNH46A, available from Stratagene; pcDNA3 available from Invitrogen; pGEX, pTrxfus, pTrc99a, pET-5, pET-9, pKK223-3, pKK233-3, pDR540, pRIT5 available from Pharmacia; and pSPORT1, pSPORT2 and pSV•SPORT1, available from Life Technologies, Inc. Other suitable vectors will be readily apparent to the skilled artisan.

Representative host cells that may be used according to the invention include, but are not limited to, bacterial cells, yeast cells, plant cells and animal cells. Preferred bacterial host cells include Escherichia spp. cells (particularly E. coli cells and most particularly E. coli strains DH10B and Stbl2), Bacillus spp. cells (particularly B. subtilis and B. megaterium cells), Streptomyces spp. cells, Erwinia spp. cells, Klebsiella spp. cells and Salmonella spp. cells (particularly S. typhimurium cells). Preferred animal host cells include insect cells (most particularly Spodoptera frugiperda Sf and Sf21 cells and Trichoplusa High-Five cells) and mammalian cells (most particularly CHO, COS, VERO, BHK and human cells). These and other suitable host cells are available commercially, for example from Life Technologies, Inc., American Type Culture Collection and Invitrogen.

In addition, the invention provides methods for producing a recombinant polypeptide, and polypeptides produced by these methods. According to this aspect of the invention, a recombinant polypeptide may be produced by culturing any of the above recombinant host cells under conditions favoring production of a polypeptide therefrom, and isolation of the polypeptide. Methods for culturing recombinant host cells, and for production and isolation of polypeptides therefrom, are well-known to one of ordinary skill in the art.

In other applications, the methods of the invention may be used to generate a gene-specific cDNA library from a complex population of poly A+ RNA. The methods of the invention, in combination with polymorphism analysis methods such as AFLP, also facilitate rapid and direct identification of transcriptional differences between two different DNA populations. Additionally, the primer-adapter used in the invention can be designed to contain a regulatory sequence, such as a promoter, enhancer or other regulatory region. In one such aspect, a promoter for T7 or SP6 RNA polymerase may be engineered into the primer-adapter, thereby enabling the production of additional copies of the original mRNA for use in amplification or subtraction. Furthermore, the methods of the invention can be used to isolate poly A+ RNA from total RNA, such as from cells, tissues, organs or organisms, or to generate a cDNA library directly from total RNA. In the latter application, the invention is particularly useful when the mRNA of interest represents only a minute fraction of the total RNA; by the invention, this low-level mRNA may be rapidly and efficiently isolated from the background of total RNA and may then be rapidly and efficiently reverse transcribed into single-stranded or double-stranded cDNA molecules for a variety of purposes such as cloning and/or amplification.

It will be readily apparent to one of ordinary skill in the relevant arts that other suitable modifications and adaptations to the methods and applications described herein are obvious and may be made without departing from the scope of the invention or any embodiment thereof. Having now described the present invention in detail, the same will be more clearly understood by reference to the following examples, which are included herewith for purposes of illustration only and are not intended to be limiting of the invention.

### Example 1

### Production and Isolation of cDNA Molecules

First and second strand cDNA synthesis reactions were conducted as described in the instruction manual for the SUPERSCRIPT Plasmid System (Life Technologies, Inc., Rockville, Md.), except that 50-5000 ng of mRNA was used as starting material to produce a library of >10⁶ clones. The primer-adapter used in cDNA synthesis contained four biotin (B) residues:

(SEQ ID NO: 1)
B-GACT(-B)AGT(-B)T(-B)CTAGATCGCGAGCGGCCGCCC(T₁₅).

Briefly, 1 μg of the biotinylated primer-adapter was used to prime first strand synthesis for 60 minutes, in a solution containing 50 mM TRIS-HCl (pH 8.3), 75 mM KCl, 3 mM MgCl₂, 10 mM DTT, 500 μM each of dATP, dCTP, dGTP and dTTP, 50 μM/ml Bio-p-A and 10,000 to 50,000 units/ml SuperScript II reverse transcriptase (Life Technologies, Inc.). Second strand synthesis was performed for two hours at 16° C. using methods described previously (Okayama, H., and Berg, P., Mol. Cell. Biol. 2:161 (1982); Gubler, U., and Hoffman, B. J., Gene 25:263 (1983); D'Alessio, J. M., et al., FOCUS 9:1 (1987)), in a solution containing 25 mM TRIS-HCl (pH 7.5), 100 mM KCl, 5 mM MgCl₂, 10 mM (NH₄)₂SO₄, 0.15 mM B-NAD+, 250 μM each of dATP, dCTP, dGTP and dTTP, 1.2 mM DTT, 65 units/ml DNA ligase, 250 units/ml DNA polymerase 1 and 13 units/ml RNase H.

During the final 30 min of the two-hour second strand cDNA synthesis reaction, streptavidin paramagnetic beads were prepared. Briefly, paramagnetic beads (Life Technologies, Inc.) were resuspended and 150 μl of bead suspension was placed into a microcentrifuge tube for each reaction. The tubes were the placed into a Magna-Sep Magnetic particle Separator (magnet) for two minutes, and supernatant removed by aspiration. The beads were then washed by adding 100 μl of TE buffer (10 mM TRIS-HCl (pH 7.5), 1 mM EDTA) to each tube, resuspending beads, and removing supernatant after two minutes as described above. Following washing, the beads were resuspended in 160 μl of Binding Buffer (10 mM Tris-HCl (pH 7.5), 1 mM EDTA, 1 M NaCl) and held at 25° C. until use in isolating cDNA.

After incubating the second strand cDNA synthesis reaction mixtures with T4 DNA polymerase, the tubes were placed on ice and the reaction terminated by the addition to each tube of 10 μl of 0.5 M EDTA. The biotinylated cDNA molecules were then isolated by contacting the solution with the streptavidin-coupled paramagnetic beads. Briefly, 160 μl of the beads prepared as described above were added to the cDNA reaction mixture tubes, and the tubes gently mixed and incubated for 60 minutes at room temperature. Tubes were then inserted into the magnet for two minutes, after which supernatants were removed and discarded. The beads were then washed by gentle resuspension with 100 μl of wash buffer (10 mM TRIS-HCl (pH 7.5), 1 mM EDTA, 500 mM NaCl), followed by re-insertion into the magnet. After two minutes, supernatants were removed and discarded and the washing step repeated. Following the second wash, beads were resuspended in 100 μl of wash buffer, transferred into fresh tubes, and washed twice as above (with five minute exposures to the magnet).

Following the second five-minute wash, supernatant was removed and discarded and cDNA molecules were removed from the beads by incubation with NotI. Briefly, 50 μl of NotI solution (41 μl of autoclaved distilled water, 5 μl of REact 3 buffer (500 mM TRIS-HCl (pH 8.0), 100 mM MgCl₂, 1 M NaCl) and 4 μl of Not I) were added to each reaction tube and tubes mixed by gentle pipetting. Tubes were incubated for two hours at 37° C., then inserted into the magnet for two minutes. Supernatants containing the cDNA molecules were withdrawn into a fresh tube, and the beads gently resuspended in 20 μl of TE buffer, re-inserted into the magnet for two minutes, and supernatants from this wash combined with those containing the cDNA molecules from above. To each tube containing pooled supernatants, 70 μl of phenol:chloroform:isoamyl alcohol (25:24:1) was added and the tubes vortexed thoroughly and centrifuged at room temperature for five minutes at 14,000×g. Following centrifugation, 65 μl of the upper, aqueous layer were removed from each tube and transferred into fresh microcentrifiige tubes, and 32 μl of 7.5 M ammonium acetate, 1 μl (20 μg) of Glycogen and 250 μl of cold (−20° C.) absolute ethanol were added to each tube. Tubes were then mixed and stored on dry ice or at −70° C. for 15 minutes, then centrifuged for 30 minutes at 14,000×g at 4° C. Supernatants were removed and discarded, 100 μl of 70% ethanol were added to the pellets and the tubes were centrifuged for two minutes at 14,000×g at room temperature. Supernatants were removed and discarded, and the pellets were dried in a speed-vac and then dissolved in TE buffer (10 μl for 50-200 ng of input mRNA, or 100 μl for 200-5000 ng of input mRNA). Final cDNA yields were determined by Cerenkov counting.

### Example 2

### Vector Ligation of cDNA and Introduction into Host Cells

From 10 to 50 ng of the cDNA was ligated into a vector (e.g., pCMVSPORT) and this ligation introduced into E. coli by transformation as described in the SUPERSCRIPT Plasmid System manual (Life Technologies, Inc.), except the cloning vector was pre-digested with NotI and SmaI. In one such ligation, 50 ng of vector was ligated to the cDNA in a 1.5 ml microcentrifuge tube with 4 μl of 5×T4 DNA ligase buffer (250 mM TRIS-HCl (pH 7.6), 50 mM MgCl₂, 5 mM ATP, 5 mM DTT, 25% (w/v) PEG-8000) and 1 μl of T4 ligase (1 unit) at 4° C. for 16 hours.

### Example 3

### cDNA Yield Comparisons

To examine the efficiency and yield of cDNA synthesis by the methods of the invention, cDNA was produced as described above and the amounts produced were compared to those obtained using an alternative commercially available system (SUPERSCRIPT Plasmid System; Life Technologies, Inc., Rockville, Md.). Briefly, after introducing the pCMV•SPORT-cDNA ligations into MAX EFFICIENCY DH5α™ and ELECTROMAX® DH10B cells, the cells were plated onto ampicillin-containing plates to determine transformation efficiencies. The cDNA inserts were sized by using the SP6 and T7 promoter primers and 40 cycles of PCR on 48 randomly chosen colonies for each experiment.

Table 1 shows a comparison of the cDNA yields obtained by the methods of the present invention to those obtained using the SuperScript Plasmid System.

TABLE 1
Comparison of the Invention to
the SUPERSCRIPT Plasmid System.

| | | | Transformants | |
|---|---|---|---|---|
| | | | per ligation | |
| | Input mRNA | Yield of | (MAX | |
| System | per reaction | cDNA | EFFICIENCY | Avg. Insert Size, |
| Tested | (ng) | (ng) | DH5α ™) | basepairs (Range) |
| Present | 1000 | 117 | 1.6 × 10⁴ | 1210 (580-2040) |
| Invention | 5000 | 619 | 2.5 × 10⁴ | 1030 (220-1810) |
| SUPER- | 1000 | 27 | 1.8 × 10⁴ | 840 (450-1400) |
| SCRIPT | 5000 | 231 | 2.0 × 10⁴ | 1280 (240-2080) |
| Plasmid | | | | |
| System | | | | |

These results demonstrate that the present invention produces about three- to four-fold greater yields of cDNA than the SUPERSCRIPT Plasmid System. Furthermore, the present invention demonstrates approximately equivalent transformation efficiencies and average insert sizes to those obtained with the SUPERSCRIPT Plasmid System. Thus, the present invention provides methods for the rapid and efficient production of full-length cDNA molecules without the use of time-consuming and yield-reducing cDNA size fractionation steps.

### Example 4

### Production and Isolation of cDNA Using Varying Amounts of Input mRNA

Having demonstrated that the methods of the invention produce cDNA rapidly and efficiently, the efficacy of the invention in producing cDNA from varying amounts of input mRNA was examined. In these studies, the amount of input mRNA was varied from 5 ng to 1 μg and the cDNA yield, transformation efficiency and average insert size determined as above. Results are shown in Table 2.

TABLE 2
Yield of cDNA Using Different Amounts of Input mRNA.

| | | Transformants | |
|---|---|---|---|
| Input mRNA | Yield | per ligation | |
| per reaction | of cDNA | (ELECTROMAX ® | Avg. Insert Size, |
| (ng) | (ng) | DH10B) | basepairs (Range) |
| 5 | 2 | 2.7 × 10⁵ | 600 (200-2000) |
| 50 | 11 | 5.1 × 10⁶ | 650 (280-1600) |
| 200 | 55 | 8.0 × 10⁶ | 930 (340-2200) |
| 1000 | 389 | 7.5 × 10⁶ | 1300 (150-2900) |

These results demonstrate that the present invention is capable of producing large cDNA libraries (i.e., >10⁵ clones) from as little as 5 ng of input mRNA. Previously, PCR (a process that biases the cDNA library) was the only method that would have enabled the production of cDNA libraries from this small amount of RNA. Together with those above, these results indicate that the invention is capable of rapidly and efficiently producing high-quality, full-length cDNA molecules from varying quantities of input mRNA, including those that show a low level of expression and thus represent only a small fraction of the polyA+ or total RNA pools.

Having now fully described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

1. A method for making a nucleic acid molecule comprising
(a) mixing a nucleic acid template with (i) one or more polypeptides having polymerase activity and/or reverse transcriptase activity and (ii) a primer adapter nucleic acid molecule˜ and
(b) incubating said mixture under conditions sufficient to make a first nucleic acid molecule complementary to all or a portion of said template,
wherein said primer-adapter nucleic acid molecule comprises one or more ligands and one or more cleavage sites.

2. The method of claim 1, wherein said first nucleic acid molecule comprises said primer-adapter nucleic acid molecule.

3. The method of claim 1, wherein said template is RNA or DNA.

4. The method of claim 3, wherein said RNA is amRNA or a polyA+ RNA molecule.

5. The method of claim 1, wherein said first nucleic acid molecule is RNA or DNA.

6. The method of claim 1, wherein said polypeptide is selected from the group consisting of a Moloney Leukemia Virus (M-MLV) reverse transcriptase, a Rous Sarcoma Virus (RSV) reverse transcriptase, an Avian Myeloblastosis Virus (AMV) reverse transcriptase, a Tne DNA polymerase, a Tma DNA polymerase, a Taq DNA polymerase, a Tth DNA polymerase, a Tli or VENT™ DNA polymerase, a Pfu or DEEPVENT™ DNA polymerase, a Pwo DNA polymerase, a Bst DNA polymerase, a Sac: DNA polymerase, a Tac DNA polymerase, a Tfl/Tub DNA polymerase, a Tru DNA polymerase, a DYNAZYME™ DNA polymerase, an Mth DNA polymerase, a Rous Associated Virus (RAV) reverse transcriptase, a Myeloblastosis Associated Virus (MAV) reverse transcriptase, a Human Immunodeficiency Virus (HIV) reverse transcriptase, a retroviral reverse transcriptase, a retrotransposon reverse transcriptase, a hepatitis B virus reverse transcriptase, a cauliflower mosaic virus reverse transcriptase, a bacterial reverse transcriptase and mutants, variants and derivatives thereof

7-9. (canceled)

10. The method of claim 1, wherein said ligand molecule is selected from the group consisting of (i) biotin; (ii) an antibody; (iii) an enzyme; (iv) lipopolysaccharide; (v) apotransferrin; (vi) ferrotransferrin; (vii) insulin; (viii) cytokines (growth factors, interleukins or colony-stimulating factors); (ix) gpI20; (x) β-actin; (xi) LFA-I; (xii) Mac-I; (xiii) glycophorin; (xiv) laminin; (xv) collagen; (xvi) fibronectin; (xvii) vitronectin; (xviii) integrins α,β and α,β; (xix) integrins α,β, α,β, α,β, α,β, α,β, α,β, and α,β; (xx) integrins α,β, α,β, α,β and α,β; (xxi) integrins α,β, α,β, α,β, α,β, α,β, α,β and α,β; (xxii) ankyrin; (xxiii) C3bi, fibrinogen or Factor X; (xxiv) ICAM-1 or ICAM-2; (xxv) spectrin or fodrin; (xxvi) CD4; (xxvii) a cytokine (e.g., growth factor, interleukin or colony-stimulating factor) receptor; (xxviii) an insulin receptor; (xxix) a transferrin receptor; (xxx) Fe+++; (xxxi) polymyxin B or endotoxinneutralizing protein (ENP); (xxxii) an enzyme-specific substrate; (xxxiii) protein A, protein G, a cell-surface Fc receptor or an antibody-specific antigen; and (xxxiv) avidin and streptavidin.

11-32. (canceled)

33. A nucleic acid molecule comprising one or more primer-adapter molecules, wherein said primer-adapter molecule comprises one or more ligands and one or more cleavage sites.

34. The nucleic acid molecule of claim 33, wherein said of said one or more cleavage sites allows removal of said one or more ligands from said nucleic acid molecule.

35. The nucleic acid molecule of claim 33, wherein said ligand is bound to one or more haptens.

36. (canceled)

37. The nucleic acid molecule of claim 33, wherein said cleavage site is a restriction endonuclease site or an endonuclease cleavage site.

38. The nucleic acid molecule of claim 33, wherein said nucleic acid molecule is double-stranded or single-stranded.

39. The nucleic acid molecule of claim 38, wherein said nucleic acid molecule is a DNA molecule, a RNA molecule, or a DNA/RNA hybrid molecule.

40. (canceled)

41. A kit for the production of a nucleic acid molecule comprising one or more containers, wherein a first container comprises a primer-adapter molecule comprising one or more ligands and one or more cleavage sites.

42. The kit of claim 41, further comprising one or more additional containers comprising one or more polypeptides having polymerase and/or reverse transcriptase activity.

43. The kit of claim 41, further comprising one or more additional containers comprising a solid support which comprises one or more haptens which specifically recognize and are capable of binding said ligand.

44-53. (canceled)


SEQUENCE LISTING (1 sequence)

SEQ ID NO: 1 - DNA (cDNA), 44 bp; single-stranded, both
  modified_base: /mod_base= OTHER /note= “Guanidine at position 1 is biotinylated”
  modified_base: /mod_base= OTHER /note= “Thymidine at position 4 is biotinylated”
  modified_base: /mod_base= OTHER /note= “Thymidine at position 7 is biotinylated”
  modified_base: /mod_base= OTHER /note= “Thymidine at position 8 is biotinylated”
gactagttctagatcgcgagcggccgcccttttttttttttttt